(19) United States
(12) Patent Application Publication
(10) Pub. No.: US 2019/0216521 A1
(43) Pub. Date: Jul. 18, 2019

(54) AN AUTOMATED SLAPHAMMER TO REMOVE ORTHOPAEDIC IMPLANTS

(71) Applicant: Pankajkumar CHHATRALA, Morbi (IN)

(72) Inventor: Pankajkumar CHHATRALA, Morbi (IN)

(21) Appl. No.: 16/334,361

(22) PCT Filed: Sep. 19, 2017
(86) PCT No.: PCT/IN2017/050413

§ 371 Date: Mar. 19, 2019

(30) Foreign Application Priority Data
Sep. 20, 2016 (IN) 201621031987

(51) Int. Cl. A61B 17/92; A61F 2/46

(52) U.S. Cl. CPC A61B 17/921; A61B 2017/00398; A61F 2/4603

(57) ABSTRACT

An automated system to remove the stuck implants/objects form bone including an adjustable shaft attached to the implant with a releasable retaining mechanism for releasably retaining a sliding weight. The automated device comprises a guide rod having a first end and a second end; a shaft member coupled to the guide rod which is movable relative to the first end of the guide rod; a sliding weight slidable along the guide rod between the first end of the guide rod and the striking member; a motor to control the device, a regulator to run machine at specific frequency of reverse impacts.

### FIELD OF THE INVENTION

The present invention relates to tools for facilitating orthopaedic procedure. The present invention relates generally to implant systems and methods for orthopaedic applications. More specifically, the present invention relates to implant extraction with coupling gear for attachment to manual and power force transducers with control over force variables. More particularly, this invention relates to the device that can be used by a surgeon to remove articles embedded in bone, such as a prosthetic, nails or implants.

### BACKGROUND OF THE INVENTION

Injuries, disease, as well as the natural aging process, can lead to changes in the musculoskeletal system of the body i.e. the bones, joints, and muscles of the body. Such changes, or injuries, can manifest in physical degeneration. To correct such damage, medical practitioners routinely perform various orthopaedic procedures.

Routine orthopaedic procedures include hip replacement and knee replacement surgery. During replacement of a joint such as the hip or knee, diseased or damaged joint surfaces are replaced with metal and plastic components shaped to allow continued use (and motion) of the joint. After incisions are made for surgical access, damaged or diseased materials, such as bones and muscles, are removed, with artificial prostheses inserted in their place. Additionally, it is often necessary to remove surgical implements that are secured to a patient. However, access to the surgical implements may be limited due to the confines of the surgical operating workplace. Apart from joints replacement, intramedullary nails (IMN) are used for internal fixation of fractured long bones. IMN is required to remove frequently because of various reasons i.e. infection, non-union of fractures, secondary fracture with IMN in place, continuous pain, psychological reasons etc.

Several different tools are used during orthopaedic procedures to place and/or remove various objects. Mallets are frequently used to apply an impacting force on a medical tool, such as a chisel, to remove bones or other implanted objects. Mallets are also commonly used to insert an implant, and to remove tools positioned in the surgical area. While mallets are effective, the impacting force must be axially applied to avoid misalignment of the prosthesis, or the inadvertent removal of bone. Moreover, the force applied must be sufficiently controlled for avoiding damage to the bone.

Slap hammers are commonly used as extraction tools for withdrawal or removal of stuck implants/objects form bone during orthopaedic surgery. This may be achieved typically by first engaging the proximal stem with a distal end of a slap hammer assembly and urging a moveable slap weight proximally against a stationary handle or slap weight. This is intended to break the bond at the stem and bone interface and eventually withdraw the prosthesis by application of sufficient axial load.

Engagement between the known slap hammer extraction tool and stem is preferably via co-operating threads, the event of a screwed stem; an extraction tool may engage via a hex nut and withdraw by opposite rotation of the stem.

Many orthopaedic procedures involve implants for replacing damaged and dysfunctional joints. For example, revision surgeries of total joint replacement (TJR) and hemi arthroplasty (replacing one-half of the joint) procedures have been developed. Hips, knees, elbows, shoulders and wrists are commonly reconstructed with implants, such as prosthetic joints that are designed for optimal wear, comfort, biocompatibility and performance. Such replacement joint implants have benefited many patients by restoring their mobility and other functions.

Stem/implant which was implanted during previous orthopaedic surgery to support bone or reconstruct normal skeletal structure (i.e. broken bone, damaged joints etc.). Over the period of time bone tissue in grows into implants in-order to supports healing bone. Because of ingrown bone tissue it is difficult to remove implant from bone.

Intra medullary Nail (IMN) is inserted to stabilize fractured long bones i.e femur, tibia. Over the period of time bone heals over IMN and bone tissue grows over IMN surface, resulting bio-bonding between bone tissue and implant surface As a result whenever it is required to remove IMN, Surgeon has to put serious effort to remove IMN from Bone. Currently Orthopaedic surgeon fix intramedullary nail with malleus like instrument and removes IMN by reverse stroking using specially designed hammer.

Orthopaedic revision procedures are necessitated in case of prosthetic/implant failures from various causes. For example, further deterioration and trauma can lead to prosthetic joint failures. Another problem relates to loosening and disengagement of the components. For example, orthopaedic cement, which is commonly used to bond prosthetic components to bone, can loosen and disengage. Looseness and “play” in implants, such as prosthetic joints, can cause significant problems. These include patient discomfort and immobility. Moreover, such looseness can increase under dynamic loading, and can ultimately lead to complications associated with implant failure.

When revision procedures are indicated by such conditions, extracting existing implants and the cement mantels bonding same can present significant difficulties. Extracting prostheses that have been permanently bonded in place with high-strength adhesives can require substantial force, with resulting trauma and collateral damage. For example, perforated and cracked existing bone structures can result from forces associated with extracting failed prostheses. Moreover, implants can become stuck during installation. For example, if the cavity formed for the implant shaft is too small, a test fit can result in immobility with resistance to both insertion and extraction. Extracting a stuck implant can require breaking the surrounding bone structure, with resulting complications.

The prior art has attempted to address some of the problems associated with orthopaedic implant extractions. For example, the Engelbrecht et al. U.S. Pat. No. 4,248,232 discloses the use of a vibrating tool to soften the cement between nested components bonded together. The Hood et al. U.S. Pat. No. 5,045,054 discloses an ultrasound power generator adapted for coupling to end prostheses and vibrating same to soften their adhesive bonds. Hood et al. disclose an ultrasonic tool for attachment to and removal of surgical components in U.S. Pat. No. 5,318,570. Vandewalle et al. U.S. Pat. No. 6,190,392 disclose an auger tool connected to an ultrasonic transducer/hand piece for extracting an osteal cement mantel. U.S. Pat. No. 5,913,860 discloses the device for impaction and extraction of nail from the bone. This device has slap and hammer which can be removed or attached to slide rod used for removal of the nail.

Several different tools have been developed to facilitate to remove stuck implants of the intramedullary nail or rod from bodies. Mallets are often used to apply an impacting force on a medical tool, such as a chisel, to remove tool from a body. Mallets are also commonly used to insert an implant, and slaphammers to remove stuck implants of the intramedullary nail or rod from bone. While slaphammers are effective, the impacting force must be axially applied to avoid misalignment of the bone, prosthesis, or the inadvertent removal of bone. Moreover, the force applied must be sufficiently accurate to avoid damages.

To overcome some of these problems, slaphammers have been developed and are widely used in orthopaedic procedures to apply an impacting force on various tools used during surgery. However, most slaphammer designs still have several drawbacks. Current slaphammers tend to be very large and heavy, and are thus difficult to handle. Exceptional care must be exercised while using these instruments to prevent injury to the patient and/or the surgeon. In particular, the surgeon's hands can be pinched between the hammer portion of the instrument and the hammer stops. Moreover, the size and weight of the slaphammer can make it very difficult for the surgeon to maintain a steady hand. The size and weight can also result in problems with storage and cleaning.

Accordingly, there remains a need for an automated slaphammer, more compact, lightweight device which can be safely and effectively used to apply extraction force to a medical instrument.

### SUMMARY OF THE INVENTION

The present invention provides a medical instrument to remove stuck implants from bone tool which is useful to accurately and safely apply a force to a medical instrument and, in particular, to a tool used during orthopaedic surgery. The automated slap hammer is designed to provide a safe and accurate procedure for applying a force to a medical device, while minimizing the risk of injury to the patient or to the surgeon's hands during use. The direct force can be directed to remove a medical tool from a location in a patient's body. In addition, the compact design of the instrument provides for ease and accuracy of use, as well as ease of cleaning and storage.

A principal object of the present invention is to provide an automated slap hammer comprising a shaft connected to implants/objects at first end and a second end assembled with, a striking assembly of the shaft rod and movable relative to the striking assembly movement and weight of striking weight. The striking assembly is an assembly of spring member 20, impactor part A 36, impactor part B 37, impacting shaft rod between the first end of the stuck implants rod and the striking member, and impactor part A 36 positioned between the striking weight and the impactor part B 37 is rotated with the help of motor 50 and gear assembly 62 attached to one end of the impactor part B 37 and other end of the impactor part B 37 having groves move the impactor part A 36 having groves and striking assembly 30 apply a proximally directed force and/or a distally directed force on stuck implants.

Another object of the present invention is to provide a method for using an automated slaphammer to apply a force to the stuck implants comprises coupling a stuck implants rod to the shaft rod 10, sliding a striking weight along the face gear 33 to impact on implants in the direction of the shaft rod 10 and against the striking assembly 30, mechanically retaining the striking weight 31 against the force tension of spring of striking assembly, and mechanically releasing the striking weight to cause the striking weight to slide along the face gear in a direction away from the stuck implants and to deliver an impact to the striking member.

Another object of the present invention is to provide, using an automated slaphammer comprises of the impactor part A 36 and impactor part B 37 is positioned adjacent to, or along the side of each other. The impactor part A 36 moves between the first end and second end of the face gear and impact away from the stuck implants. The impactor part A 36 and shaft road are connected with striking spring. The impactor part B 37 is rotated by motor 50 and impactor part A 36 is slidably movable between the face gear 33 first end and second end of the face gear slot.

Another object of the present invention, includes apparatus, systems, methods, and the like which constitute part of the invention, will become more apparent upon reading the following detailed description of the exemplary embodiments and viewing the drawings.

Another object of the present invention provides an improved orthopaedic device to remove stuck implants such as intramedullary nail or rod and the instruments associated therewith.

Another object of the present invention is an automated system that helps surgeon to deliver controlled force to implant in order to remove it from bone safely.

Another object of the present invention provides an improved orthopaedic device wherein the grooves in the device controls the force exerted to remove stuck implants of the intramedullary nail or rod.

Another object of the present invention provides an improved orthopaedic device with a motor attachment to control revolution per minute of the device.

Another object of the present invention provides an improved orthopaedic device to control the striking force by adjusting spring force.

### BRIEF DESCRIPTION OF THE DRAWINGS

The drawings constitute a part of this invention and include exemplary embodiments of the present invention illustrating various objects and features thereof.

FIG. 1. is the schematic view of slap hammer

FIG. 2. is the external view of slaphammer

FIG. 3 is the cross-sectional view of the slaphammer

FIG. 4 is the horizontal view of the slaphammer

FIG. 5 is the cross sectional view of grove assembly

FIG. 6 is the exploded view of an automated slaphammer withdrawal or removal tool mated to a medical instrument on stuck implants/objects.

### DETAILED DESCRIPTION OF THE INVENTION

Reference will now be made in detail to exemplary embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the exemplary embodiments and methods. The invention according to its various aspects is particularly pointed out and distinctly claimed in the attached claims read in view of this specification, and appropriate equivalents

The present invention provides a medical instrument automated slaphammer which is useful to accurately and safely apply a force to a medical instrument and, in particular, to a tool used during withdrawal or removal of stuck implants/objects form Bone during orthopaedic surgery. The instrument is designed to provide a safe and accurate procedure for applying a force to a medical device, while minimizing the risk of injury to the patient or to the surgeon's hands during use. The withdrawal or removal force can be directed to withdraw a stem/implant which was implanted during previous orthopaedic surgery to support bone or reconstruct normal skeletal structure (i.e broken bone, damaged joints) from a location in a patient's body, to prepare an implant site using a broaching device or similar bone preparation device.

The present invention provides an automated system that helps the surgeon to deliver controlled extraction force to implant in order to remove it from bone and femoral stems in revision hip surgery.

The present invention provides an automated tool used to apply force towards or away from a surgical area in a joint replacement procedure is known as “automated slap hammer” or “automated slide hammer”. Slap hammers, also known by the alternate name of a “slide hammer”, provide a weight capable of sliding about a guide rod or face gear. The sliding weight can be used to generate a force when the sliding weight reaches and strikes a stop provided on the guide rod or the face gear. For example, a slap hammer tool can be used to provide force downward towards an object, to provide precision force (e.g., impact) towards a point of interest at an end of the slap hammer. Likewise, a slap hammer tool can be used to provide force away from a stuck implants rod, such as to extract ans tuck implants rod coupled to an shaft rod 10 end of the slap hammer tool.

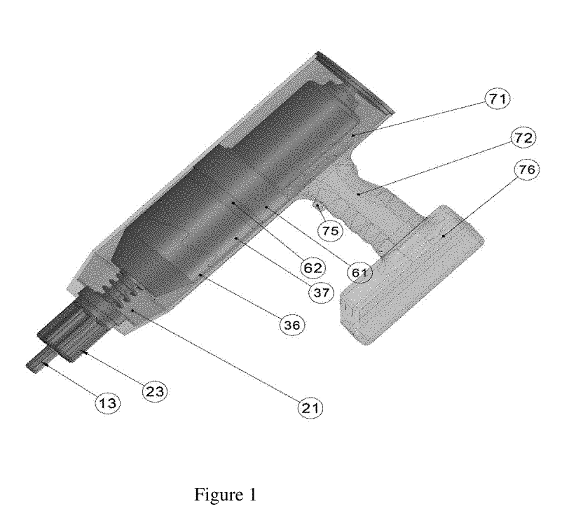

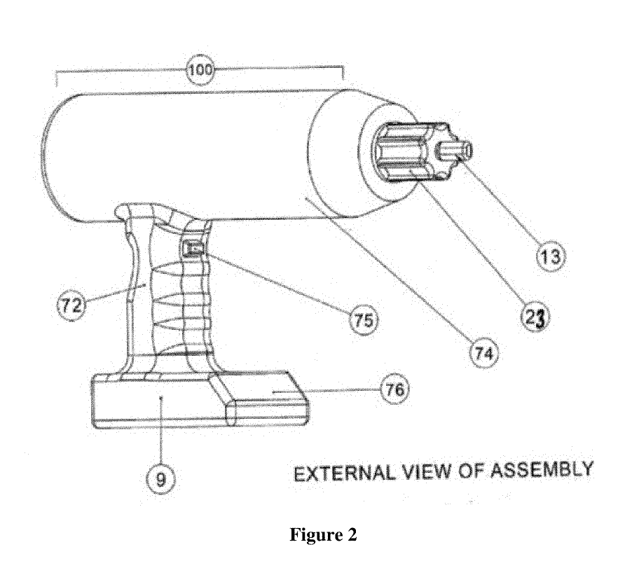

FIGS. 1 and 2 is an illustration of a slap hammer device 100 according to an embodiment of the invention. The slap hammer 100 includes a screw coupling mechanism 13 which will be coupled to the surgical implant 70 (not shown). The coupling mechanism 13 is joined to the knob 23 which is internally connected to spring member/spring tensioner/spring groove member 20 which is used to adjust the tension of the spring element 21 of the slaphammer, by rotating tension member through 23. The body of the slaphammer 74 consists of the striking assembly 30, grove assembly 38 and the gear assembly 62. The handle of the slaphammer 72 consists of a stand 9, button 75 and battery pack 76 which also act as counter balance while operating instrument. The slaphammer can be operated electrically, manually or by solar energy. The striking assembly 30 consists of striking weight 31, impactor part A 36 and impactor part B 37. The striking assembly 30 is connected to the gear assembly 62 with an intermediate disc 61 which is attached to the motor 50.

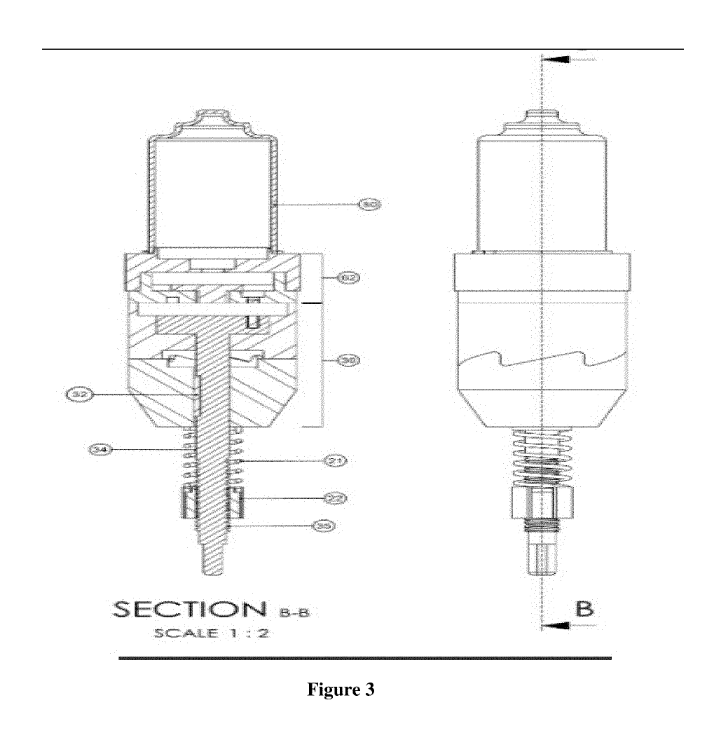

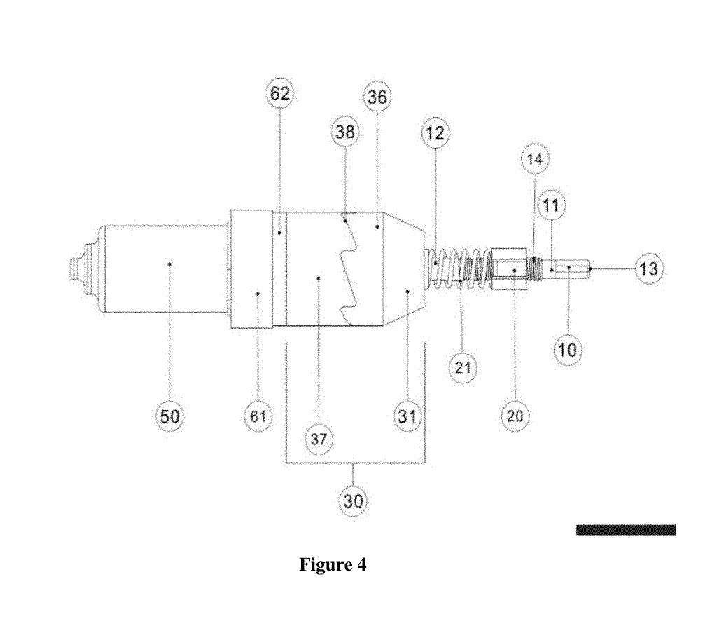

FIG. 3 and FIG. 4 illustrates cross sectional and horizontal view of slap hammer. The slaphammer 100 generally includes a shaft rod 10 having a threaded portion 14 arranged on a distal end 11 and a spring element 21 arranged on a proximal end 12 with striking assembly 30. The spring member/spring tensioner/spring groove member 20 will engage with the knob 23 to adjust the tension of the spring element 21. The spring member, spring tensioner or spring groove member can be used in the embodiment of the invention. The striking assembly 30 consists of striking weight 31, impactor part A 36 and impactor part B 37. The striking assembly 30 is connected to the gear assembly 62 which is attached to the motor 50. When motor drives impactor part B 37 in rotational movement, impactor part A 36 moves axially because of key and slot 32 mentioned in FIG. 3. Thus the key slot maintains the axial movement of the rod. The zig zag groove assembly 38 also maintains the movement of the shaft rod 10. A spring member 20, illustrated in FIG. 3 as a spring element 21, is positioned about the face gear 33 at the second end 35 of face gears 33. The spring element 21 comprises a cylindrical compression spring designed to resist applied compression force and to store energy in a compressed stance. The spring member 20 may comprise other mechanisms, such as a tensioning spring. As the striking weight 31 is moved toward the second end 35 of the face gear 33, the spring element 21 is compressed to store energy and striking weight 31 in the opposite direction, towards the first end 34 of face gears 33.

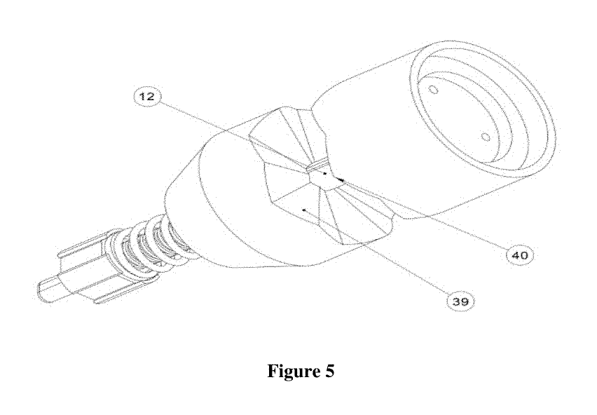

FIG. 5 illustrates cross sectional view of the grooves of the of slap hammer, which is a zig-zag assembly maintaining the axial movement of the shaft rod. When an operator wants to activate the slap hammer, the triggering mechanism can be triggered by rotating impactor part B 37 and displacing the grove B 40 over grove A 39 sliding impactor part A 36 and the along the guide rod 33. As the triggering mechanism moves the striking assembly 30 and impactor part A 36 impacts the shaft rod 10 at the distal end 11 attached to the stuck implants rod. As used herein “a grove” is a long and narrow indentation built into a material, generally for the purpose of allowing another material or part to move within the groove and be guided by it. It is however to be noted that the shape of the groove does not limit the scope of invention.

Devices according to embodiments of the present invention may be fabricated from light, strong and rigid biocompatible materials. For example, in some embodiments, the slap hammer may comprise metal, metal alloys, polymeric composites or other known suitable materials. In some embodiments, various components or the entire slap hammer may be disassembled or taken apart for storage and/or cleaning. In some embodiments, biasing elements of differing strengths, or tensions, may be utilized, to provide varying forces on objects coupled to the slap hammer.

Various slap hammers may be configured in many different dimensions, and deliver a wide range of impact forces. In some embodiments, the slap hammer may be dimensioned for delivering substantially large impact forces and in other embodiments, the slap hammer may be dimensioned for delivering lesser impact forces. Advantageously, the adjustable striking assembly 30 allows slap hammers 100 of any dimension to deliver a wide range of impact forces.

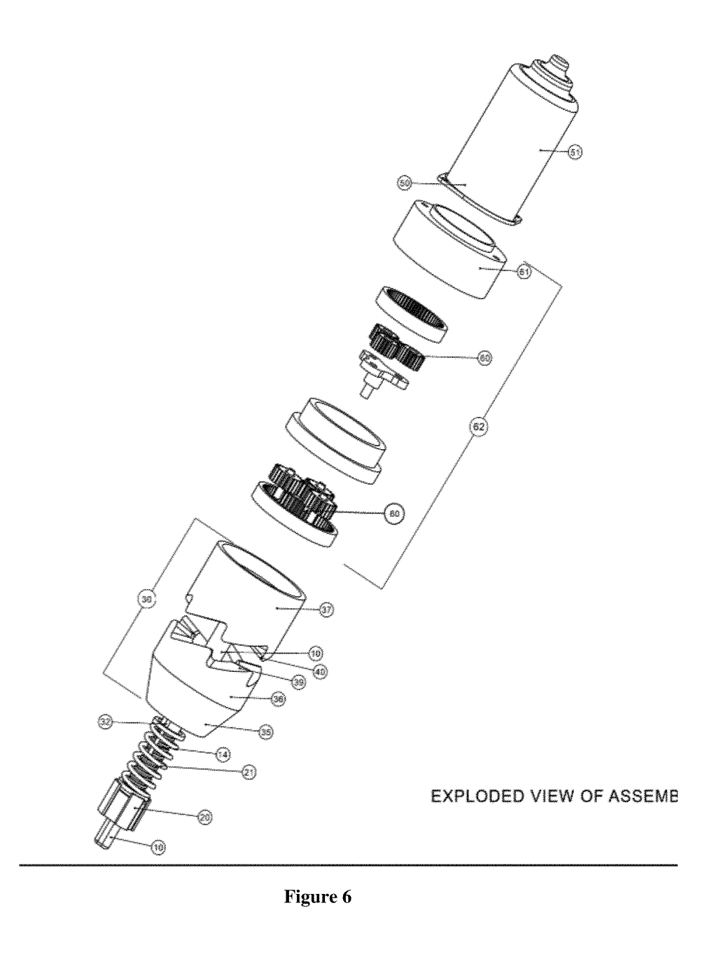

With initial reference to FIG. 6, an exploded view of slap hammer for in-vivo assembly of implants according to the present teachings is shown and generally identified at reference. The slaphammer 100 generally includes a shaft rod 10 having a threaded portion 14 arranged on a distal end 11 and a spring member 20 arranged on a proximal end 12 with striking assembly 30. The slap hammer 100 also includes striking weight 31 that is slidable along zig zag face gears groove assembly 38 and spring member 14. The slap hammer 100 is used to apply a force to stuck implants rod coupled to the shaft rod 10. Shaft 10 consists of a key slot with key 32, which restricts rotation of Impactor A 36 but allows the axial movement.

The shaft rod 10 can be coupled to an object, such as a surgical implant, prosthesis, or IMN, in a variety of ways using coupling mechanism 13. As shown in FIG. 6, the threaded portion 14 of the shaft rod 10 comprises a screw coupling mechanism 13. The coupling mechanism 13 comprises a threaded end configured to mate with a threaded portion of a surgical implement. Other coupling mechanisms include pliers, a hook, a clamp, a ring, a magnet, and like coupling mechanisms. Still other coupling mechanisms may be used, such as a snap fit or friction fit connection. In some scenarios, a removable or interchangeable coupling mechanism may be desirable, so that the slap hammer may be quickly disconnected from one surgical implement and connected to another. In other embodiments a more permanent coupling is used to provide greater stability and rigidity.

As shown in FIG. 6, the striking weight 31 moves, or slides, along zig zag groove of the face gears 38 via a key slot 32 in the striking weight 31. Face gears 38 extends through the key slot 32, and the striking weight can move toward spring member 20 and away from the first end 34 of face gears 38.

A spring member 20, illustrated in FIG. 6 as a spring element 21, is positioned about the face gear 33 at the second end 35 of face gears groove assembly 38. The spring element 21 comprises an axial compression spring designed to resist applied compression force and to store energy in a compressed stance. The spring member 20 may comprise other mechanisms, such as a tensioning spring. As the striking weight 31 is moved toward the second end 35 of the rod 33, the spring element 21 is compressed to store energy and striking weight 31 in the opposite direction, towards the first end 34 of face gears 38.

A releasable retaining mechanism, retains the striking weight 31 against the action of the spring element 21. A groove mechanism 38 for mechanically triggering the release of impactor part A 36 and the striking weight 31 is coupled to the impactor part B 37. The groove mechanism 38 consist of groves A39 on impactor part A 36 and groves B 40 on impactor part B 37 mechanically trigger, releasing the striking weight 31 which is propelled by the spring element 21 in the opposite direction, towards the first end 34 of the face gear 33 towards the impactor part B 37.

The striking weight 31 is adapted to rotate along face gears 38 between the spring element 21 and impactor part B 37 with releasable retaining mechanism and slides the striking weight 31 between the first ends 34 at the second end 35 of face gears 38. The spring element 14 is disposed between the striking weight 31 and the shaft rod llproximal end 12, the spring element 21 releases and urges the striking weight 31 in the direction of the impactor part B 37. The striking weight 31 may be urged against the action of the spring 21 and locked in place via the releasable retaining mechanism with impactor part B 37.

When an operator wants to activate the slap hammer, the triggering mechanism can be triggered by rotating impactor part B 37 and displacing the grove B 40 over grove A 39 sliding impactor part A 36 and the along the face gears 33. As the triggering mechanism moves the striking assembly 30 and impactor part A 36 impacts the shaft rod 10 at the distal end 11 attached to the stuck implants rod. The striking weight 31 of impactor part A 36 assembled moves towards the spring member 20 at proximal end 12 of the shaft rod 11. The spring member 20 store potential energy in the form of a compression spring will compress and store potential energy. The spring member 20 is attached to the spring knob 23 and spring element 21 which maintains the spring member 20. The striking weight 31 of impactor part A 36 is released in the direction towards impactor part B 37 with great impact on shaft rod 11 at the distal end 18 attached to the stuck implants rod. The impactor part B 37 is rotated with the help of motor 50 and gear assembly 62 attached to one end of the motor 50 and other end of the impactor part B 37 having groves B move the impactor part A 36 having groves A and striking assembly apply a proximally directed force and/or push the striking weight 31 upward a distally directed force on stuck implants towards the spring member 20 which adjusts the compression of the spring element 21.

FIG. 6 which is is a exploded view of a slap hammer according to another embodiment of the invention. In FIG. 6, a slap hammer comprises a zig zag face 38 having a first end 34 and a second end 35. The slap hammer also includes a striking weight 31 that slides along the face gear 33. Face gears 38 of the slap hammer shown in further comprises a coupling mechanism in the form of coupling mechanism 13. The coupling mechanism 13 can be screwed, clamped, or hooked clamp on an object to be removed from a surgical area. Once the coupling mechanism 13 are screwed, clamped, or hooked to an object, the striking weight 31 can be released at the first end 34 of the face gear, and propelled by the tension of the spring element 21 towards the second end 35 of face gears 38, where it will impact the striking assembly 30, and impart a pulling force on the object coupled by the coupling mechanism 13 with shaft rod 10.

FIG. 6 is an illustration of various connecting mechanisms according to embodiments of the invention. The first end 34 of the shaft rod 10 can be configured to accept a variety of different connecting mechanisms, such as screw, clamped or pliers. Other off-the-shelf and/or custom made products can be used. As shown in FIG. 6, other connecting mechanisms comprise various threaded screw endings. Alternatively, various connecting mechanisms comprise pin connectors which are advantageous for clamping around smaller objects, such as wire.

FIG. 6 is an illustration of a using a slap hammer according to another embodiment of the invention. The method of coupling mechanism 13 by coupling a shaft rod to an object. The shaft rod 10 may be coupled to the object, i.e. the target workpiece, via a coupling mechanism, such as a threaded screw, pliers, a clamp, a ring, or a hook. In other embodiments, other coupling mechanisms may be used, such as a magnet, or a vacuum suction coupling mechanism. The object coupled to the guide rod may be surgical instrument, or some other object.

The force applied to the object may be controlled by optionally adjusting the position of a spring member 20 on the shaft rod 10 by rotating knob 23. The striking assembly 30 is positioned on a second end of the shaft rod 10 with spring member 20 and the object coupled to the shaft rod 10 (i.e. the target workpiece) on the first end of the shaft rod 10. By moving the impactor part B 37 the striking assembly 30 relative to the first end of the face gear, the acceleration of the striking weight 31 may be increased or decreased by adjusting the spring member 20 by controlling the spring tension force, and thus the force delivered by the striking weight 31 may be adjusted. The striking assembly 30 can be adapted to move along the gears toward or away from the first end of the face gear 33 in order to adjust the position of the striking assembly 24 relative to the striking weight.

After the shaft rod 10 is coupled to the object 70 and the acceleration of the striking weight 31 is adjusted, the impactor part B 37 is rotated, striking assembly 30 slid, along the face gear 33 towards the object and against the spring member 20. As the sliding weight is slide towards the object 70, the spring member 20 store potential energy. For example, spring member 20 in the form of a compression spring will compress and store potential energy.

While or immediately after the striking weight 31 is positioned against the spring member 20, the striking weight 31 is mechanically retained against the spring member 20. A temporary locking mechanism, i.e. a releasable retaining mechanism, releasable retains the striking weight 31 against the spring member 20.

Finally, the striking weight 31 is mechanically released from the temporary locking mechanism, causing the striking weight 31 to slide away from the object along the face gear 33 to deliver an impact to the shaft rod 10. A triggering mechanism may mechanically release the striking weight 31 from the temporary locking mechanism. When the striking weight 31 impacts the shaft rod 10, the slap hammer moves or jerks in the same direction traveled by the striking weight, potentially moving or dislodging the object coupled to the slap hammer in the same direction.

As it is described in the diagram below, implant is attached to shaft of the device with specific attachment. The surgeon now applies traction to handle, and starts machine by using regulator.

The regulator button 75 allows the surgeon to run machine at specific frequency of reverse impacts. The present invention is an automated system wherein the surgeon can apply more force to implant compared to hammer, face gears and rod.

The presently disclosed slap hammer provides numerous advantages in the art. For example, the disclosed slap hammer allows a user to have greater control over the force of the hammer strike than in conventional slap hammers. Specifically, disclosed slap hammers can deliver variable forces, allowing a single device to deliver a range of controlled impact forces.

This invention is susceptible to considerable variation in its practice. Therefore the foregoing description is not intended to limit, and should not be construed as limiting the invention to the particular exemplifications presented hereinabove. Rather, what is intended to be covered is as set forth in the ensuing claims and the equivalents thereof permitted as a matter of law.

1. An automated slap hammer comprising:
a) Shaft 10 connected to implants at first end and a second end assembled with, a striking assembly 30;
b) a striking assembly 30, with spring member 20, impactor part A 36, impactor part B 37, impacting shaft rod between the first end of the stuck implants rod and the striking member, and impactor part A 36 positioned between the striking weight and the impactor part B 37
c) impactor part B 37, is rotated with the help of motor 50 and gear assembly 62 attached to one end of the impactor part B 37 and other end of the impactor part B 37 having grooves move the impactor part A 36 having grooves and striking assembly 30 apply a proximally directed force to a distally directed force on stuck implants;
d) groove mechanism 38 at one end of the impactor part A 36, impactor part B 37.

2. An automated slap hammer according to claim 1 further comprising a screw coupling mechanism 13 at the first end of face gear 33 coupled to the spring member 20.

3. An automated slap hammer according to claim 1, wherein the spring member comprises of the spring element 21 and the knob 23.

4. An automated slap hammer according to claim 1, threaded portion 14 of the shaft rod 10 comprises a screw coupling mechanism 13.

5. An automated slap hammer according to claim 4, wherein, threaded end configured to mate with a threaded portion of a surgical implant.

6. An automated slap hammer according to claim 5 wherein, shaft rod 10 coupled to an object, such as a surgical implant, prosthesis, or IMN, in a variety of ways using coupling mechanism 13.

7. An automated slap hammer according to claim 1, wherein the button is used to start, stop, and control striking frequency of slap hammer.

8. An automated slap hammer according to claim 1, wherein the groove assembly consists of the grooves which maintain the axial movement of Impactor A 36.

9. An automated slap hammer according to claim 1, wherein the groove assembly consists of the grooves in the device controls the force exerted to remove stuck implants of the intramedullary nail or rod.

10. An automated slap hammer according to claim 1 wherein, Shaft 10 consists of a key slot with key 32, which restricts rotation of Impactor A 36 but allows the axial movement.

11. An automated slap hammer according to claim 1 comprising g the striking member 30, wherein the striking member consists of striking weight 31 guides the face gears 33 to impact on implants in the direction of the shaft rod 10 and against the striking assembly 30, mechanically retaining the striking weight 31 against the force tension of spring member 20 of striking assembly 30, and mechanically releasing the striking weight 31 to cause the striking weight 31 to slide along the face gear 33 in a direction away from the stuck implants and to deliver an impact to the striking member.

12. An automated slap hammer according to claim 1, wherein the force to the stuck implants comprises:
a. coupling a stuck implants rod to the shaft rod 10;
b. sliding a striking weight along the face gear 33 to impact on implants in the direction of the shaft rod 10 and against the striking assembly 30;
c. mechanically retaining the striking weight 31 against the force tension of spring of striking assembly and;
d. mechanically releasing the striking weight to cause the striking weight to slide along the face gear in a direction away from the stuck implants and to deliver an impact to the striking member.

13. An automated slap hammer according to claim 1 wherein the striking assembly comprises:
a. the impactor part A 36 and impactor part B 37 is positioned adjacent along the side of each other;
b. The impactor part A 36 moves between the first end and second end of the face gear 33 and impact away from the stuck implants;
c. The impactor part A 36 and shaft road are connected with striking spring;
d. The impactor part B 37 is rotated by motor 50 and impactor part A 36 is axially movable between the face gear 33 first end and second end of the face gear 33 slot.
